(19) United States
(12) Patent Application Publication
(10) Pub. No.: US 2026/0294823 A1
(43) Pub. Date: Oct. 1, 2026

(54) ANTIDIABETIC PHARMACEUTICAL COMPOSITION

(71) Applicant: Imam Abdulrahman Bin Faisal University, Dammam (SA)

(72) Inventors: Suhailah Saud ALJAMEEL, Dammam (SA); Kamal Adel AMIN, Dammam (SA)

(73) Assignee: Imam Abdulrahman Bin Faisal University, Dammam (SA)

(21) Appl. No.: 19/299,459

(22) Filed: Aug. 14, 2025

Related U.S. Application Data
(60) Provisional application No. 63/781,789, filed on Apr. 1, 2025.

(51) Int. Cl. A61K 9/51; A61K 38/28; A61P 3/10

(52) U.S. Cl. CPC A61K 9/51; A61K 9/5161; A61K 9/5192; A61K 38/28; A61P 3/10

(57) ABSTRACT

A pharmaceutical composition includes therapeutic nanoparticles including a core including insulin, a shell comprising chitosan, the shell disposed on the core, and a coating containing pectin and dextrin on the shell, and a pharmaceutically acceptable carrier and/or excipient. A method of forming the pharmaceutical composition includes adding a sodium tri-polyphosphate solution to a chitosan solution including the chitosan and acetic acid while stirring to form a shell solution, mixing a dextrin solution with an insulin solution to form a core solution, forming a pectin solution, mixing the shell solution, the core solution, and the pectin solution using a homogenizer to form a precursor mixture, aging the precursor mixture to form the nanoparticles, and mixing the nanoparticles with the pharmaceutically acceptable carrier and/or excipient.

### CROSS REFERENCE TO RELATED APPLICATION(S)

This application claims the benefit of U.S. Provisional Application No. 63/781,789, filed Apr. 1, 2025, which is incorporated by reference in its entirety.

### STATEMENT OF PRIOR DISCLOSURE BY AN INVENTOR

Aspects of the present disclosure are described in Ramadan, H.; Moustafa, N.; Ahmed, R. R.; et al. (2024) “Therapeutic effect of oral insulin-chitosan nanobeads pectin-dextrin shell on streptozotocin-diabetic male albino rats.” Heliyon, 10, e35636, and Mohammed, S. R.; Abdel-Moneim, A.; Abdel-Reheim, E. S.; et al. (2024) “De novo fabrication of oral insulin-loaded chitosan/dextrin/pectin nanospheres and their antidiabetic efficacy in streptozotocin-induced diabetic rats.” Journal of Optoelectronic and Biomedical Materials, 16(2), 73-87, both of which are incorporated herein by reference in their entireties.

### STATEMENT OF ACKNOWLEDGEMENT

Support provided by the College of Science at Imam Abdulrahman Bin Faisal University Dammam, Saudi Arabia is gratefully acknowledged.

### BACKGROUND

### Technical Field

The present disclosure relates to the field of anti-diabetic drugs. More particularly, the present disclosure pertains to a pharmaceutical composition incorporating insulin, a chitosan shell, and a coating, delivered via pharmaceutical grade excipient.

### Description of Related Art

The “background” description provided herein is for the purpose of generally presenting the context of the disclosure. The work of the presently named inventors, to the extent it is described in this background section, as well as aspects of the description which may not otherwise qualify as prior art at the time of filing, are neither expressly nor impliedly admitted as prior art against the present invention.

Type 1 diabetes mellitus (T1DM) is characterized by the autoimmune destruction of pancreatic β-cells, resulting in insufficient or absent insulin production and persistent hyperglycemia [T. Tomita, Apoptosis of pancreatic β-cells in Type 1 diabetes, Biomolecules & Biomedicine, 17 (3) (2017) 183-189, and S. E. Kahn, Y.-C. Chen, N. Esser, A. J. Taylor, D. H. van Raalte, S. Zraika, C. B. Verchere, The β cell in diabetes: integrating biomarkers with functional measures, Endocr. Rev. 42 (5) (2021) 528-583]. Streptozotocin (STZ), a compound commonly used to induce experimental diabetes, exerts cytotoxic effects on β-cells through mechanisms involving reactive oxygen species (ROS) generation and DNA alkylation [Y. Nomier, G. F. Asaad, A. Salama, M. E. Shabana, S. Alshahrani, M. Firoz Alam, T. Anwer, S. Sultana, Z. Rehman, A. Khalid, Explicit mechanistic insights of Prosopis juliflora extract in streptozotocin-induced diabetic rats at the molecular level, Saudi Pharmaceut. J. 31 (10) (2023) 101755]. Hyperglycaemia itself contributes to oxidative stress, which is increasingly recognized as a central mechanism underlying the pathogenesis of diabetes and its complications [C. M. O. Volpe, P. H. Villar-Delfino, P. M. F. Dos Anjos, J. A. Nogueira-Machado, Cellular death, reactive oxygen species (ROS) and diabetic complications, Cell Death Dis. 9 (2) (2018) 119]. Inflammatory responses mediated by T cells and cytokine release further exacerbate β-cell damage and impair insulin secretion [M. Ande ̌l, V. Ne ̌mcov\acute{ }a, N. Pavlíkov\acute{ }a, J. Urbanov\acute{ }a, M. Cech\acute{ }akov\acute{ }a, A. Havlov\acute{ }a, R. Strakov\acute{ }a, L. Vec ̌er ̌ov\acute{ }a, V. Mandys, J. Kov\acute{ }ar ̌, P. Heneberg, J. Trnka, J. Pol\acute{ }ak, (Factors causing damage and destruction of beta-cells of the islets of Langerhans in the pancreas), Vnitr. Lek. 60 (9) (2014) 684-690 and M. K. Prasad, S. Mohandas, K. M. Ramkumar, Dysfunctions, molecular mechanisms, and therapeutic strategies of pancreatic β-cells in diabetes, Apoptosis 28 (2023) 958-976].

Elevated glucose and free fatty acid levels intensify oxidative cellular injury by promoting free radical formation and diminishing antioxidant defences [K. N. Keane, V. F. Cruzat, R. Carlessi, P. I. de Bittencourt Jr., P. Newsholme, Molecular events linking oxidative stress and inflammation to insulin resistance and β-cell dysfunction, Oxid. Med. Cell. Longev. 2015 (2015) 181643]. Transcription factors such as NF-κB p65 and SIRT-1 play important roles in regulating genes associated with apoptosis, inflammation, immune responses, and cellular metabolism [E. Casper, the crosstalk between Nrf2 and NF-κB pathways in coronary artery disease: can it be regulated by SIRT6? Life Sci. 330 (2023) 122007, and H. Iskender, E. Dokumacioglu, T. M. Sen, I. Ince, Y. Kanbay, S. Saral, The effect of hesperidin and quercetin on oxidative stress, NF-κB and SIRT1 levels in a STZ-induced experimental diabetes model, Biomed. Pharmacother. 90 (2017) 500-508]. Additionally, the Nrf2-mediated upregulation of heme oxygenase-1 (HO-1) provides cytoprotective effects against oxidative stress [P. Newsholme, K. N. Keane, R. Carlessi, V. Cruzat, Oxidative stress pathways in pancreatic β-cells and insulin-sensitive cells and tissues: importance to cell metabolism, function, and dysfunction, Am. J. Physiol. Cell Physiol. 317 (3) (2019) C420-C433].

Current therapeutic strategies for T1DM primarily rely on exogenous insulin administration. However, subcutaneous insulin injections are associated with significant limitations, including the risk of hypoglycaemia, poor glycaemic control, and the inability to mimic the physiological insulin secretion of pancreatic β-cells [V. Pathak, N. M. Pathak, C. L. O'neill, J. Guduric-Fuchs, R. J. Medina, Therapies for type 1 diabetes: current scenario and future perspectives, Clin. Med. Insights Endocrinol. Diabetes 12 (2019)]. These limitations often result in acute and chronic complications, including coma, ketosis, and long-term vascular damage.

Recent research has explored oral insulin delivery systems as a non-invasive alternative to injections. However, challenges such as enzymatic degradation and poor intestinal absorption continue to hinder their clinical efficacy [M. Wang, C. Wang, S. Ren, J. Pan, Y. Wang, Y. Shen, Z. Zeng, H. Cui, X. Zhao, Versatile oral insulin delivery nanosystems: from materials to nanostructures, Int. J. Mol. Sci. 23 (6) (2022) 3362]. Natural polysaccharide-based nano-carriers have emerged as promising candidates for oral drug delivery due to their biocompatibility, biodegradability, and ability to enhance drug stability and absorption [A. Abdel-Moneim, H. Ramadan, Novel strategies to oral delivery of insulin: current progress of nanocarriers for diabetes management, Drug Dev. Res. 83 (2) (2022) 301-316]. Chitosan, pectin, and dextran are among the most studied polysaccharides for this purpose. Chitosan exhibits mucoadhesive properties and facilitates paracellular transport [V. Mikus ̌ov\acute{ }a, P. Mikus ̌, Advances in chitosan-based nanoparticles for drug delivery, Int. J. Mol. Sci. 22 (17) (2021) 9652, and E. B. Souto, S. B. Souto, J. R. Campos, P. Severino, T. N. Pashirova, L. Y. Zakharova, A. M. Silva, A. Durazzo, M. Lucarini, A. A. Izzo, A. Santini, Nanoparticle delivery systems in the treatment of diabetes complications, Molecules 24 (23) (2019) 4209], while pectin and dextran offer antioxidant, immunomodulatory, and protective effects against enzymatic degradation [S. T. Minzanova, V. F. Mironov, D. M. Arkhipova, A. V. Khabibullina, L. G. Mironova, Y. M. Zakirova, V. A. Milyukov, Biological activity and pharmacological application of pectic polysaccharides: a review, Polymers 10 (12) (2018) 1407, W. M. Kedir, E. M. Deresa, T. F. Diriba, Pharmaceutical and drug delivery applications of pectin and its modified nanocomposites, Heliyon 8 (9) (2022) e10654, and Q. Hu, Y. Lu, Y. Luo, Recent advances in dextran-based drug delivery systems: from fabrication strategies to applications, Carbohydr. Polym. 264 (2021) 117999].

Despite the foregoing advancements, there remains a pressing requirement for improved oral insulin delivery systems that may effectively regulate blood glucose levels, mitigate oxidative stress, and preserve pancreatic tissue integrity. The development of such systems is important to address the limitations of current therapies and to improve the quality of life for individuals with T1DM. Accordingly, one object of the present disclosure is to provide a pharmaceutical composition configured to be orally deliverable, that may circumvent the above specified drawbacks and limitation of the materials and methods known in the art.

### SUMMARY

In an exemplary embodiment, a pharmaceutical composition is described. The pharmaceutical composition includes therapeutic nanoparticles including a core including insulin, a shell including chitosan, the shell disposed on the core, and a coating including pectin and dextrin disposed on the shell, and a pharmaceutically acceptable carrier and/or excipient.

In some embodiments, the nanoparticles have a mean particle size of 25 nanometers (nm) to 250 nm.

In some embodiments, the core further includes dextrin.

In some embodiments, the dextrin is derived from corn having a reducing sugar content of less than 5 wt. % based on a total weight of dextrin.

In some embodiments, the pectin is pectin derived from citrus peel having a Galacturonic acid of at least 74.0 wt. % based on a total weight of pectin.

In some embodiments, the chitosan has a mean molecular weight of 190,000 to 300,000 Daltons (Da).

In some embodiments, the pharmaceutically acceptable carrier and/or excipient is at least one selected from the group consisting of a buffer, an inorganic salt, a synthetic fatty acid, a vegetable oil, a fatty ester, a surfactant, and a polymer.

In another exemplary embodiment, a method of forming the pharmaceutical composition is described. The method includes adding a sodium tri-polyphosphate solution to a chitosan solution including the chitosan and acetic acid while stirring to form a shell solution, mixing a dextrin solution with an insulin solution to form a core solution, forming a pectin solution, mixing the shell solution, the core solution, and the pectin solution using a homogenizer to form a precursor mixture, aging the precursor mixture at 4° C. for 24 to 72 hours to form the nanoparticles, and mixing the nanoparticles with the pharmaceutically acceptable carrier and/or excipient.

In some embodiments, the dextrin solution is formed by heating a mixture of dextrin and water to 100° C. and cooling to room temperature.

In some embodiments, the chitosan solution has a chitosan concentration of 0.1 to 1% w/v and a pH of 3.5 to 6.0.

In some embodiments, the dextrin solution has a dextrin concentration of 0.25 to 2.5% w/v.

In some embodiments, the insulin solution has an insulin concentration of 100 to 1000 micrograms per milliliter (μg/mL).

In some embodiments, the pectin solution has a pectin concentration of 2.5 to 7.5 w/v.

In some embodiments, the method has an insulin encapsulation efficiency of 60 to 80% based on a total amount of insulin present in the core solution.

In some embodiments, the method has an insulin drug loading of 17.5 to 37.5% based on a total amount of insulin present in the core solution.

In some embodiments, the pharmaceutically acceptable carrier and/or excipient is at least one selected from the group consisting of a buffer, an inorganic salt, a synthetic fatty acid, a vegetable oil, a fatty ester, a surfactant, and a polymer.

In yet another embodiment, a method of treating diabetes is described. The method includes administering to a subject in need thereof an effective amount of the pharmaceutical composition described above.

In some embodiments, the diabetes is at least one selected from the group consisting of STZ-induced diabetes and Type 1 diabetes.

In some embodiments, the pharmaceutical composition is administered in an amount of 10 to 100 IU of nanoparticles per kg of bodyweight.

In some embodiments, the pharmaceutical composition is administered orally.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates an exemplary flow chart depicting a method of forming a pharmaceutical composition for treating type-1 diabetes mellitus, according to certain embodiments.

FIG. 1B illustrates a schematic diagram of an animal model and experimental design, according to certain embodiments.

FIG. 2 illustrates a field emission scanning electron microscopy (FESEM) image showing surface morphology of the pharmaceutical composition (INS-CsNBs-PD shell), according to certain embodiments.

FIG. 3 illustrates a high resolution transmission electron microscopy (HR-TEM) image depicting an internal structure of the pharmaceutical composition (INS-CsNBs-PD shell), according to certain embodiments.

FIG. 4 illustrates a bar graph showing initial and final body weights (IB.wt. and FB.wt.) of animal subjects in different groups, with statistical significance indicated by superscript notations, according to certain embodiments.

FIG. 5 illustrates fasting and postprandial blood glucose levels following treatment in various groups where the values are expressed as mean±SEM, where n=6, FBGL refers to fasting blood glucose levels (F=58.298, P<0.05), and PBGL refers to final postprandial blood glucose levels (F=78.990, P<0.05), according to certain embodiments.

FIG. 6A illustrates a photomicrograph of the pancreatic section of the control group (C) displaying normal pancreatic acini and an intact islet of Langerhans with peripheral alpha cells and centrally located n-cells, according to certain embodiments.

FIG. 6B illustrates a pancreatic section of an untreated diabetic group (D) exhibiting cytoplasmic vacuolation (V), cellular degeneration, pyknosis, necrosis of n-cells, mitotic figures, and a shrunken degenerated islet, according to certain embodiments.

FIG. 6C illustrates another pancreatic section of the diabetic group (D) showing karyomegaly in acinar cells, apoptosis, small shrunken islets, and vascular congestion, according to certain embodiments.

FIG. 6D illustrates the pancreatic section of the group treated orally with INS-CsNBs-PD shell (F), showing partial regeneration of n-cells and improvement in degenerative features, according to certain embodiments.

FIG. 6E illustrates the pancreatic section of the diabetic group treated with oral free insulin (FO), showing islet shrinkage, degeneration, congestion, and cytoplasmic vacuolation, according to certain embodiments.

FIG. 6F illustrates the pancreatic section of the diabetic group treated with blank nano-formula (NB), showing small islets without notable degenerative changes, according to certain embodiments.

FIG. 6G illustrates the pancreatic section of the group treated with subcutaneous insulin (Sc), showing islets with regenerative activity, increased islet volume, and reappearance of n-cells, according to certain embodiments.

FIG. 7A illustrates a photomicrograph of the pancreatic section of the control group (C) showing β-cells within the islet exhibiting strong immunoreactivity for insulin, according to certain embodiments.

FIG. 7B illustrates the pancreatic section of the untreated diabetic group (D) showing a marked reduction in insulin expression due to extensive degradation of β-cells, according to certain embodiments.

FIG. 7C illustrates the pancreatic section of the group treated orally with INS-CsNBs-PD shell (F), exhibiting low insulin immunoreactivity associated with a small number of β-cells, according to certain embodiments.

FIG. 7D illustrates the pancreatic section of the group treated with oral free insulin (FO), displaying reduced islet size and diminished insulin immunoreactivity due to decreased β-cell count, according to certain embodiments.

FIG. 7E illustrates the pancreatic section of the group treated orally with CsNBs-PD shell (NB), showing decreased insulin expression in β-cells, according to certain embodiments.

FIG. 7F illustrates the pancreatic section of the group injected with subcutaneous insulin (Sc), presenting increased insulin expression in regenerated β-cells, according to certain embodiments.

FIG. 7G illustrates morphometric analysis of the mean area percentage of insulin-immunoreactive β-cells (μm²) in different groups, where statistical significance is indicated with superscript notations compared to control and diabetic groups, according to certain embodiments.

FIG. 8A illustrates oxidative stress biomarker levels including MDA, SOD, and GST in pancreatic tissue homogenates across different experimental groups, where data are expressed as mean±SE with statistical significance denoted by superscript notations, according to certain embodiments.

FIG. 8B illustrates antioxidant biomarker levels including CAT, GPx, and GSH in pancreatic tissue homogenates of the experimental groups, according to certain embodiments.

FIG. 9A illustrates an effect of the INS-CsNBs-PD shell nano-formula on serum insulin and C-peptide levels in control and STZ-induced diabetic rats, according to certain embodiments.

FIG. 9B illustrates the effect of the INS-CsNBs-PD shell nano-formula on serum IL-1B and IL-6 levels in control and STZ-induced diabetic rats, according to certain embodiments.

FIG. 10 illustrates the gene expression levels of Bax, Bcl2, Nrf2, and HO-1 across different experimental groups, according to certain embodiments.

FIG. 11 illustrates the protein expression levels of NF-κB P65 and SIRT-1 in various experimental rat groups, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, reference numerals designate identical or corresponding parts throughout several views. Further, as used herein, the words ‘a’, ‘an’ and the like generally carry a meaning of ‘one or more,’ unless stated otherwise.

Furthermore, the terms ‘approximately’, ‘approximate’, ‘about’, and similar terms generally refer to ranges that include the identified value within a margin of 20%, 10%, or preferably 5%, and any values therebetween.

A ‘pharmaceutical composition’ refers to a mixture of the compounds described herein or pharmaceutically acceptable salts, esters, or prodrugs thereof, with other chemical components, such as physiologically acceptable carriers and excipients.

‘Pharmaceutically acceptable salt’ or ‘pharmaceutically acceptable ester’ refers to a compound in a pharmaceutically acceptable form such as an ester, a phosphate ester, a salt of an ester, or a related) which, upon administration to a subject in need thereof, provides the compound of Formula (I) described herein. Pharmaceutically acceptable salts and esters retain the biological effectiveness and properties of the free bases, which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. Suitable salts include those derived from alkali metals such as potassium and sodium, and alkaline earth metals such as calcium and magnesium, among numerous other acids well-known in the art.

As used herein, a ‘pharmaceutically acceptable carrier’ refers to a carrier or diluent that does not cause significant irritation to an organism and does not abrogate the biological activity and properties of the compound of Formula (I). The term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

An ‘excipient’ refers to an inert substance added to a pharmaceutical composition to facilitate the administration of a compound further. Examples, without limitation, of excipients include calcium carbonate, calcium phosphate, various sugars and types of starch, cellulose derivatives, gelatin, vegetable oils, and polyethylene glycols.

As used herein, the term ‘nanoparticles (NPs)’ refers to particles having a particle size of 1 nm to 500 nanometers (nm) within the scope of the present invention. The NPs may exist in various morphological shapes, such as nanotubes, nanowires, nanospheres, nanocrystals, nanorectangles, nanotriangles, nanopentagons, nanohexagons, nanoprisms, nanodisks, nanocubes, nanoribbons, nanoblocks, nanobeads, nanotoroids, nanodiscs, nanobarrels, nanogranules, nanowhiskers, nanoflakes, nanofoils, nanopowders, nanoboxes, nanostars, tetrapods, nanobelts, nano-urchins, nanoflowers, and mixtures thereof.

As used herein, the term ‘buffer’ refers to a solution that resists changes in pH upon the addition of small amounts of acid or base.

As used herein, the term ‘inorganic salt’ refers to a chemical compound composed of positively charged ions (cations) and negatively charged ions (anions) held together by ionic bonds, typically resulting from the neutralization of an acid and a base.

As used herein, the term ‘synthetic fatty acid’ refers to a fatty acid that is chemically synthesized, often through processes like hydroformylation or ozonolysis, rather than being derived from natural sources.

As used herein, the term ‘vegetable oil’ refers to a triglyceride extracted from plant sources, primarily seeds or fruits, and is typically liquid at room temperature.

As used herein, the term ‘fatty ester’ refers to an ester compound formed by the reaction of a fatty acid with an alcohol, resulting in the elimination of water.

As used herein, the term ‘surfactant’ refers to a substance that lowers the surface tension between two liquids or between a liquid and a solid, facilitating processes like emulsification, wetting, or foaming.

A surfactant molecule includes one or more hydrophilic head units attached to one or more hydrophobic tails. The tail of most surfactants includes a hydrocarbon chain, which can be branched, linear, or aromatic. Fluorosurfactants have fluorocarbon chains. Siloxane surfactants have siloxane chains. Gemini surfactant molecules include two or more hydrophilic heads and two or more hydrophobic tails. Many surfactants include a polyether chain terminating in a highly polar anionic group. The polyether groups often include ethoxylated (polyethylene oxide-like) sequences inserted to increase the hydrophilic character of a surfactant. Alternatively, polypropylene oxides may be inserted to increase the lipophilic character of a surfactant.

Cationic surfactants have cationic functional groups at their head, such as primary and secondary amines. The cationic surfactants include octenidine dihydrochloride; cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB). A cationic surfactant may be replaced by a nonionic surfactant, an anionic surfactant, a cationic surfactant, a viscoelastic surfactant, or a zwitterionic surfactant.

Anionic surfactants contain anionic functional groups at their head, such as sulfate, sulfonate, phosphate, and carboxylate. The anionic surfactant may be an alkyl sulfate, an alkyl ether sulfate, an alkyl ester sulfonate, an alpha olefin sulfonate, a linear alkyl benzene sulfonate, a branched alkyl benzene sulfonate, a linear dodecylbenzene sulfonate, a branched dodecylbenzene sulfonate, an alkyl benzene sulfonic acid, a dodecylbenzene sulfonic acid, a sulfosuccinate, a sulfated alcohol, a ethoxylated sulfated alcohol, an alcohol sulfonate, an ethoxylated and propoxylated alcohol sulfonate, an alcohol ether sulfate, an ethoxylated alcohol ether sulfate, a propoxylated alcohol sulfonate, a sulfated nonyl phenol, an ethoxylated and propoxylated sulfated nonyl phenol, a sulfated octyl phenol, an ethoxylated and propoxylated sulfated octyl phenol, a sulfated dodecyl phenol, and an ethoxylated and propoxylated sulfated dodecyl phenol. Other anionic surfactants include ammonium lauryl sulfate, sodium lauryl sulfate (sodium dodecyl sulfate, SLS, or SDS), and related alkyl-ether sulfates sodium laureth sulfate (sodium lauryl ether sulfate or SLES), sodium myreth sulfate, docusate (dioctyl sodium sulfosuccinate), perfluorooctanesulfonate (PFOS), perfluorobutanesulfonate, alkyl-aryl ether phosphates, and alkyl ether phosphates.

Zwitterionic (amphoteric) surfactants have both cationic and anionic groups attached to the same molecule. The zwitterionic surfactants include CHAPS (3-[(3-cholamidopropyl)dimethylammonio]-1-propanesulfonate), cocamidopropyl hydroxysultaine, ocamidopropyl betaine, phospholipids, and sphingomyelins.

Nonionic surfactants have a polar group that does not have a charge. These include long chain alcohols that exhibit surfactant properties, such as cetyl alcohol, stearyl alcohol, cetostearyl alcohol, oleyl alcohol, and other fatty alcohols. Other long chain alcohols with surfactant properties include polyethylene glycols of various molecular weights, polyethylene glycol alkyl ethers having the formula CH₃—(CH₂)₁₀₋₁₆—(O—C₂H₄)₁₋₂₅—OH, such as octaethylene glycol monododecyl ether and pentaethylene glycol monododecyl ether; polypropylene glycol alkyl ethers having the formula: CH₃—(CH₂)₁₀₋₁₆—(O—C₃H₆)₁₋₂₅—OH; glucoside alkyl ethers having the formula CH₃—(CH₂)₁₀₋₁₆—(O-glucoside)₁₋₃-OH, such as decyl glucoside, lauryl glucoside, octyl glucoside; polyethylene glycol octylphenyl ethers having the formula C₈H₁₇—(C₆H₄)—(O—C₂H₄)₁₋₂₅—OH, such as Triton X-100; polyethylene glycol alkylphenyl ethers having the formula C₉H₁₉—(C₆H₄)—(O—C₂H₄)₁₋₂₅—OH, such as nonoxynol-9; glycerol alkyl esters such as glyceryl laurate; polyoxyethylene glycol sorbitan alkyl esters such as polysorbate, sorbitan alkyl esters, cocamide MEA, cocamide DEA, dodecyldimethylamine oxide, block copolymers of polyethylene glycol and polypropylene glycol, such as poloxamers, and polyethoxylated tallow amine (POEA).

A dendritic surfactant molecule may include at least two lipophilic chains that have been joined at a hydrophilic center and have a branch-like appearance. In each dendritic surfactant, there may be from about 2 lipophilic moieties independently to about 4 lipophilic moieties attached to each hydrophilic group, or up to about 8 lipophilic moieties attached to the hydrophilic group for example. ‘Independently’ as used herein with respect to ranges means that any lower threshold may be combined with any upper threshold. The dendritic surfactant may have better repulsion effect as a stabilizer at an interface and/or better interaction with a polar oil, as compared with other surfactants. Dendritic surfactant molecules are sometimes called ‘hyperbranched’ molecules.

A dendritic extended surfactant is a dendritic surfactant having a non-ionic spacer arm between the hydrophilic group and a lipophilic tail. For example, the non-ionic spacer-arm extension may be the result of polypropoxylation, polyethoxylation, or a combination of the two with the polypropylene oxide next to the tail and polyethylene oxide next to the head. The spacer arm of a dendritic extended surfactant may contain from about 1 independently to about 20 propoxy moieties and/or from about 0 independently to about 20 ethoxy moieties. Alternatively, the spacer arm may contain from about 2 independently up to about 16 propoxy moieties and/or from about 2 independently up to about 8 ethoxy moieties. ‘Independently’ as used herein with respect to ranges means that any lower threshold may be combined with any upper threshold. The spacer arm extensions may also be formed from other moieties including, but not necessarily limited to, glyceryl, butoxy, glucoside, isosorbide, xylitols, and the like. For example, the spacer arm of a dendritic extended surfactant may contain both propoxy and ethoxy moieties. The polypropoxy portion of the spacer arm may be considered lipophilic; however, the spacer arm may also contain a hydrophilic portion to attach the hydrophilic group. The hydrophilic group may generally be a polyethoxy portion having about two or more ethoxy groups. These portions are generally in blocks, rather than being randomly mixed. Further, the spacer arm extension may be a poly-propylene oxide chain.

Another type of surfactant is a viscoelastic surfactant (VES). Conventional surfactant molecules are characterized by having one long hydrocarbon chain per surfactant head-group. In a viscoelastic gelled state these molecules aggregate into worm-like micelles. A viscoelastic gel is a gel that has elastic properties, meaning that the gel at least partially returns to its original form when an applied stress is removed. Typical viscoelastic surfactants include N-erucyl-N,N-bis(2-hydroxyethyl)-N-methyl ammonium chloride and potassium oleate, solutions of which form gels when mixed with inorganic salts such as potassium chloride and/or with organic salts such as sodium salicylate. Previously described surfactants may also be considered viscoelastic surfactants.

As used herein, the term ‘polymer’ refers to a large molecule composed of repeating structural units, typically connected by covalent chemical bonds, and can be of natural or synthetic origin.

As used herein, the term ‘diabetes’ refers to a chronic condition characterized by elevated blood glucose levels resulting from insufficient insulin production or ineffective utilization of insulin by the body.

As used herein, the term ‘STZ-induced diabetes’ refers to a model of diabetes in animals induced by the administration of streptozotocin (STZ), a compound that selectively damages insulin-producing beta cells in the pancreas, leading to hyperglycemia.

As used herein, the term ‘Type 1 diabetes’ refers to an autoimmune disorder where the body's immune system attacks and destroys the insulin-producing beta cells in the pancreas, resulting in little to no insulin production.

As used herein, ‘analogue’ or ‘analog’ refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic, or it may have altered reactivity as compared to the parent compound. The analogue may mimic the chemical and/or biologically active of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers.

As used herein, ‘derivative’ refers to a chemically or biologically modified version of a chemical compound that is structurally similar to a parent compound and (actually or theoretically) derivable from that parent compound. A ‘derivative’ differs from an ‘analogue’ in that a parent compound may be the starting material to generate a ‘derivative,’ whereas the parent compound may not necessarily be used as the starting material to generate an ‘analogue.’ A derivative may or may not have different chemical or physical properties of the parent compound. For example, the derivative may be more hydrophilic, or it may have altered reactivity as compared to the parent compound. Derivatization (i.e., modification) may involve the substitution of one or more moieties within the molecule (e.g., a change in a functional group). The term ‘derivative’ also includes conjugates, and prodrugs of a parent compound (i.e., chemically modified derivatives that can be converted into the original compound under physiological conditions).

As used herein, ‘international unit (IU)’ refers to a standardized measure used to quantify the biological activity or effect of a substance, such as a vitamin, hormone, or enzyme.

As used herein, ‘insulin encapsulation efficiency (EE)’ refers to the percentage of insulin successfully encapsulated within a delivery system (such as nanoparticles, microspheres, or liposomes) relative to the total amount of insulin used in the formulation.

As used herein, ‘drug loading’ refers to the amount of a therapeutic agent incorporated into a delivery system, such as nanoparticles or liposomes.

Aspects of the present disclosure are directed to an oral insulin delivery system including insulin-loaded chitosan nanobeads (INS-CsNBs) coated with a pectin-dextrin (PD) shell. This system is designed to enhance the bioavailability and therapeutic efficacy of insulin in managing diabetes mellitus, particularly in models induced by streptozotocin (STZ). The system aims to protect insulin from enzymatic degradation in the gastrointestinal tract and facilitate its absorption, thereby improving glycemic control. Upon oral administration, the PD-coated nanobeads are intended to release insulin in a controlled manner, enhancing its therapeutic effects. The system's performance is evaluated through various biochemical and histopathological analyses, including measuring blood glucose levels, antioxidant enzyme activities, inflammatory markers, and the expression of specific proteins and genes associated with oxidative stress and apoptosis in pancreatic tissue. Additionally, immunohistochemical staining is employed to visualize insulin granules within pancreatic β-cells. The findings indicate that the oral administration of INS-CsNBs with a PD shell offers a promising alternative to traditional subcutaneous insulin injections, providing a more convenient and patient-friendly approach to diabetes management.

A pharmaceutical composition includes therapeutic nanoparticles including a core including insulin. The core further includes dextrin. The dextrin is derived from corn having a reducing sugar content of less than 5 wt. % based on the total weight of dextrin. Dextrin may be derived from various starch sources beyond corn, including wheat, potato, tapioca, rice, sorghum, cassava, oat, arrowroot, sweet potato, barley, rye, millet, sago, teff, kudzu root, acorn, breadfruit, canna, Colocasia, chestnut, water chestnut, yam, fava bean, lentil, mung bean, pea, chickpea, soybean, coconut, and plantain.

The therapeutic nanoparticles include a shell including chitosan. The chitosan has a mean molecular weight of 190,000 to 300,000 Daltons (Da), preferably from 190,500 to 194,500 Da, preferably from 191,000 to 194,000 Da, preferably from 191,500 to 193,500 Da, preferably from 192,000 to 193,000 Da, and preferably from 192,500 to 192,500 Da. The molecular weight ranges from 195,000 to 200,000 Da, preferably from 195,500 to 199,500 Da, preferably from 196,000 to 199,000 Da, preferably from 196,500 to 198,500 Da, preferably from 197,000 to 198,000 Da, and preferably from 197,500 to 198,000 Da, preferably 200,000 Da. The shell is disposed on the core. The therapeutic nanoparticles include a coating including pectin and dextrin disposed on the shell. The pectin is pectin derived from citrus peel having a Galacturonic acid of at least 74.0 wt. % based on the total weight of pectin. A pharmaceutical composition includes a pharmaceutically acceptable carrier and/or excipient. The pharmaceutically acceptable carrier and/or excipient is at least one selected from the group consisting of a buffer, an inorganic salt, a synthetic fatty acid, a vegetable oil, a fatty ester, a surfactant, and a polymer. The nanoparticles have a mean particle size of 25 to 250 nm, preferably 35 to 235 nm, preferably 40 to 230 nm, preferably 45 to 220 nm, preferably 50 to 215 nm, preferably 55 to 210 nm, preferably 60 to 205 nm, preferably 65 to 200 nm, preferably 70 to 195 nm, preferably 75 to 190 nm, preferably 80 to 185 nm, preferably 85 to 180 nm, preferably 90 to 175 nm, preferably 95 to 170 nm, preferably 100 nm.

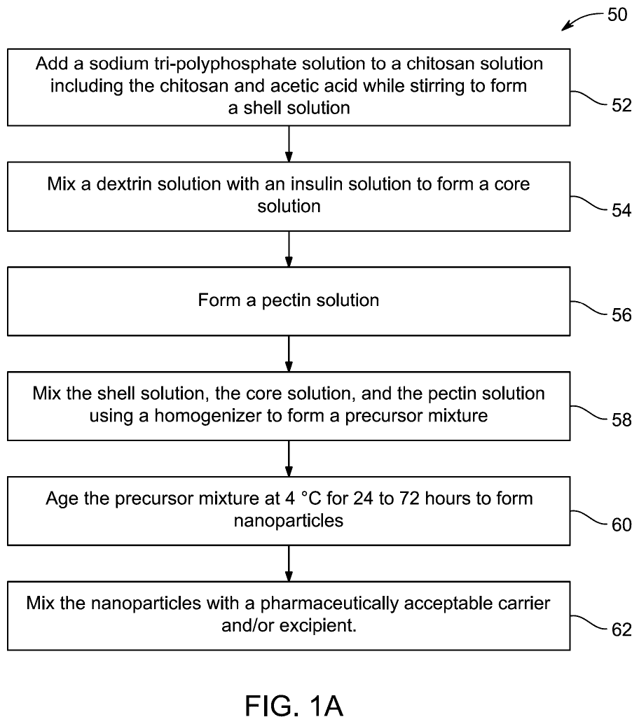

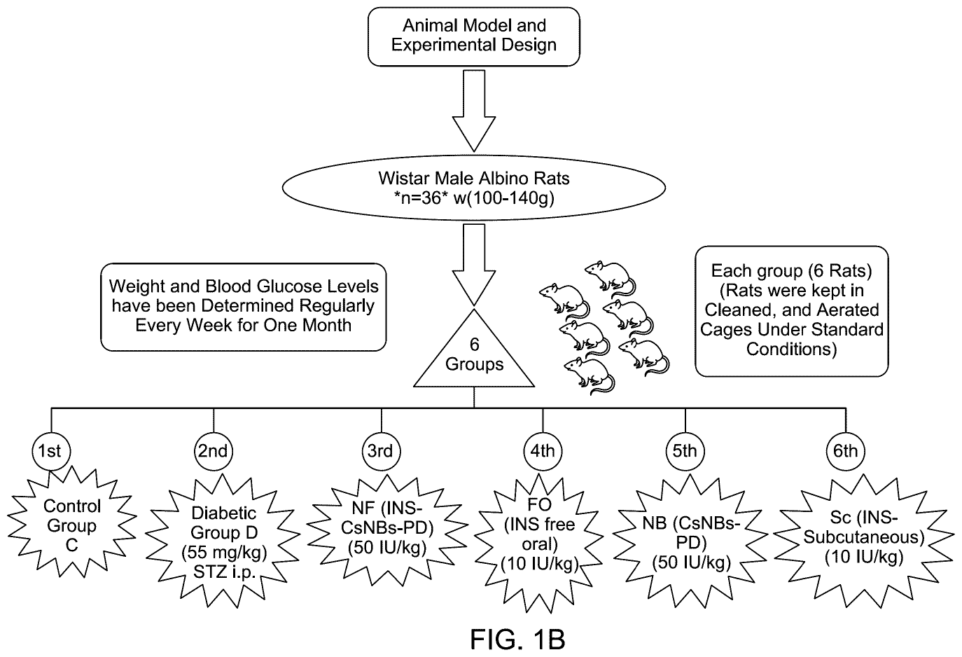

FIG. 1A illustrates a schematic flow chart of a method 50 of forming the pharmaceutical composition. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes adding a sodium tri-polyphosphate solution to a chitosan solution including the chitosan and acetic acid while stirring to form a shell solution. Sodium hexametaphosphate, polyvinyl alcohol (PVA), citric acid may be used as substitutes for the sodium tri-polyphosphate solution, chitosan and acetic acid, respectively. In some embodiments, sodium tri-polyphosphate solution is added drop-wise to the chitosan solution.

At step 54, the method 50 includes mixing a dextrin solution with an insulin solution to form a core solution. In some embodiments, maltodextrin, sorbitol, PVA, sodium chloride, polyvinylpyrrolidone (PVP), calcium chloride, albumin, dextran, chitosan may also be added to the core solution.

At step 56, the method 50 includes forming a pectin solution.

At step 58, the method 50 includes mixing the shell solution, the core solution, and the pectin solution using a homogenizer to form a precursor mixture. In some embodiments, high shear mixers, ultrasonic homogenizers (sonicators), microfluidizers, rapidojet technology, conventional agitators with premixing, paddle blenders, mortar and pestle, handheld immersion blenders, overhead stirring mixers, vortex mixers may also be used as an alternative to homogenizer.

At step 60, the method 50 includes aging the precursor mixture at 4° C. for 24 to 72 hours, preferably 24 to 71 hours, preferably 25 to 70 hours, preferably 26 to 69 hours, preferably 27 to 68 hours, preferably 28 to 67 hours, preferably 29 to 66 hours, preferably 30 to 65 hours, preferably 31 to 64 hours, preferably 32 to 63 hours, preferably 33 to 62 hours, preferably 34 to 61 hours, preferably 35 to 60 hours, preferably 36 to 59 hours, preferably 37 to 58 hours, preferably 38 to 57 hours, preferably 39 to 56 hours, preferably 40 to 55 hours, preferably 41 to 54 hours, preferably 42 to 53 hours, preferably 43 to 52 hours, preferably 44 to 51 hours, preferably 45 to 50 hours, preferably 46 to 49 hours, preferably 48 hours to form the nanoparticles. As used herein, the term ‘aging’ refers to the deliberate process of allowing a precursor mixture to stand under controlled conditions typically at a low temperature such as 4° C. for a specified duration, generally between 24 to 72 hours. This period enables the components within the solution to undergo chemical and physical changes that enhance the homogeneity and stability of the mixture.

At step 62, the method 50 includes mixing the nanoparticles with the pharmaceutically acceptable carrier and/or excipient. In some embodiments, the mixing can be done by stirring, swirling, sonicating, or a combination thereof. In a preferred embodiment, mixing of the nanoparticles with the pharmaceutically acceptable carrier and/or excipient is performed using a magnetic stirrer.

The dextrin solution is formed by heating a mixture of dextrin and water to 100° C. and cooling to room temperature. In some embodiments, the heating can be performed by using heating appliances such as ovens, microwaves, autoclaves, hot plates, heating mantles and tapes, oil baths, salt baths, sand baths, air baths, hot-tube furnaces, and hot-air guns, muffle furnace, tube furnace, electric oven, or fluidized bed furnace.

The chitosan solution has a chitosan concentration of 0.1 to 1%, preferably 0.2% to 0.8%, preferably 0.22% to 0.7%, preferably 0.24% to 0.6%, preferably 0.25% to 0.5%, preferably 0.26% to 0.4%, preferably 0.27% to 0.38%, preferably 0.28% to 0.35%, preferably 0.29% to 0.32%, preferably 0.3%, weight per volume (w/v) and a pH of 3.5 to 6.0, preferably 3.6 to 5.9, preferably 3.7 to 5.8, preferably 3.8 to 5.7, preferably 3.9 to 5.6, preferably 4.0 to 5.5, preferably 4.1 to 5.4, preferably 4.2 to 5.3, preferably 4.3 to 5.2, preferably 4.4 to 5.1, preferably 4.8.

The dextrin solution has a dextrin concentration of 0.25 to 2.5%, w/v, preferably 0.3%, w/v to 2.4%, w/v, preferably 0.4%, w/v to 2.3%, w/v, preferably 0.5%, w/v to 2.2%, w/v, preferably 0.6%, w/v to 2.1%, w/v, preferably 0.7%, w/v to 2.0%, w/v, preferably 0.8%, w/v to 1.9%, w/v, preferably 0.9%, w/v to 1.8%, w/v, preferably 1.0%, w/v.

The insulin solution has an insulin concentration of 100 to 1000 micrograms per milliliter (μg/mL), preferably 150 μg/mL to 950 μg/mL, preferably 200 μg/mL to 900 μg/mL, preferably 250 μg/mL to 850 μg/mL, preferably 300 μg/mL to 800 μg/mL, preferably 350 μg/mL to 750 μg/mL, preferably 400 μg/mL to 700 μg/mL, preferably 450 μg/mL to 650 μg/mL, preferably 500 μg/mL. The pectin solution has a pectin concentration of 2.5 to 7.5%, w/v, preferably 3.0%, w/v to 7.0%, w/v, preferably 3.5%, w/v to 6.5%, w/v, preferably 3.6%, w/v to 5%, w/v, preferably 3.7%, w/v to 5%, w/v, preferably 3.8%, w/v to 5%, w/v, preferably 3.9 to 5%, w/v, preferably 4%, w/v.

The method has an insulin encapsulation efficiency of 60 to 80%, preferably 65% to 80%, preferably 69±2.75% based on a total amount of insulin present in the core solution. The method has an insulin drug loading of 17.5 to 37.5%, preferably 22.5% to 32.5%, preferably 27.5% to 27.5%, preferably 26.28±0.56% based on a total amount of insulin present in the core solution.

A method of treating diabetes is described. The method includes administering to a subject in need thereof an effective amount of the pharmaceutical composition. ‘Subject’ refers to a living organism. Non-limiting examples include humans, other mammals, dogs, cats, bovines, rats, mice, dogs, monkeys, goat, sheep, cows, and other non-mammalian animals. In embodiments, a subject is a cat or a dog. In embodiments, a subject is a mammal. In embodiments, a subject is a primate. In embodiments, a subject is human. The diabetes is at least one selected from the group consisting of STZ-induced diabetes and Type 1 diabetes. The pharmaceutical composition is administered in an amount of 10 to 100 IU, preferably 15 to 95 IU, preferably 20 to 90 IU, preferably 25 to 85 IU, preferably 30 to 80 IU, preferably 35 to 75 IU, preferably 40 to 70 IU, preferably 45 to 65 IU, preferably 50 IU of nanoparticles per kg of bodyweight. The pharmaceutical composition is administered orally.

A pharmaceutical composition of the present disclosure can then be administered orally, systemically, parenterally, by inhalation spray, rectally, or topically in dosage unit formulations containing conventional non-toxic pharmaceutically acceptable carriers, adjuvants, and vehicles as desired. In some embodiments, the method of administration of the composition or an analogue or derivative thereof is oral. In other embodiments, the compound or an analogue or derivative thereof is administered by injection, such as, for example, through a peritumoral injection.

Topical administration can also involve the use of transdermal administration, such as transdermal patches or iontophoresis devices. The term parenteral, as used herein, includes intravesical, intradermal, transdermal, subcutaneous, intramuscular, intralesional, intracranial, intrapulmonary, intracardial, intrasternal, and sublingual injections, or infusion techniques. Formulation of drugs is discussed in, for example, Hoover, John E., Remington's Pharmaceutical Sciences, Mack Publishing Co., Easton, Pa.; 1975. Another example includes Liberman, H. A. and Lachman, L., Eds., Pharmaceutical Dosage Forms, Marcel Decker, New York, N.Y., 1980, which is incorporated herein by reference in its entirety.

Injectable preparations, for example, sterile injectable aqueous or oleaginous suspensions, can be formulated according to the known art using suitable dispersing or wetting agents and suspending agents. The sterile injectable preparation can also be a sterile injectable solution or suspension in a nontoxic, parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any fixed oil can be employed, including synthetic mono- or diglycerides. In addition, fatty acids such as oleic acid find use in the preparation of injectables. Dimethyl acetamide, surfactants including ionic and non-ionic detergents, and polyethylene glycols can be used. Mixtures of solvents and wetting agents, such as those discussed above, are also useful. Suppositories for rectal administration of the compound or an analogue or derivative thereof can be prepared by mixing the steroid or an analogue or derivative thereof with a suitable non-irritating excipient such as cocoa butter, synthetic mono- di- or triglycerides, fatty acids, and polyethylene glycols that are solid at ordinary temperatures but liquid at the rectal temperature and will therefore melt in the rectum and release the drug.

Solid dosage forms for oral administration can include capsules, tablets, pills, powders, and granules. In such solid dosage forms, the compounds of this disclosure are ordinarily combined with one or more adjuvants appropriate to the indicated route of administration. If administered per oz, a contemplated steroid or an analogue or derivative thereof can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the active compound in hydroxypropyl methylcellulose. In the case of capsules, tablets, and pills, the dosage forms can also include buffering agents such as sodium citrate, magnesium or calcium carbonate, or bicarbonate. Tablets and pills can additionally be prepared with enteric coatings.

### EXAMPLES

The following examples demonstrate a pharmaceutical composition and a method of preparation thereof. The examples are provided solely for illustration and are not to be construed as limitations of the present disclosure, as many variations thereof are possible without departing from the spirit and scope of the present disclosure.

### Example 1: Materials

Streptozotocin (STZ), chitosan (Cs) with medium molecular weight, dextrin derived from corn (D2006), pectin (P9135 25G 100G), and sodium tripolyphosphate (TPP) were procured from Sigma-Aldrich Co., Missouri, USA. Commercially available insulin (100 IU/mL) was obtained from Novo Nordisk. Glacial acetic acid, sodium hydroxide, and all other reagents used were of analytical grade and sourced from standard commercial suppliers. Kits for the determination of malondialdehyde (MDA), catalase (CAT), superoxide dismutase (SOD), glutathione peroxidase (GPx), and reduced glutathione (GSH) were purchased from BioDiagnostic Co., Dokki, Giza, Egypt.

### Example 2: Synthesis of INS-Loaded Chitosan Nanobeads Coated with Pectin-Dextrin Shell (INS-CsNBs-PD Shell)

An improved ionic gelation technique was employed to synthesize insulin-loaded chitosan nanobeads coated with a pectin-dextrin shell (INS-CsNBs-PD shell) [S. Seyam, N. A. Nordin, M. Alfatama, Recent progress of chitosan and chitosan derivatives-based nanoparticles: pharmaceutical perspectives of oral insulin delivery, Pharmaceuticals 13 (10) (2020) 307, incorporated herein by reference in its entirety]. Chitosan was dissolved at a concentration of 0.3% (w/v) in deionized water containing 1% acetic acid, adjusted to pH 4.8. A chitosan-to-sodium tripolyphosphate (TPP) mass ratio of 4:1 was used. Nanoparticle formation was initiated by the dropwise addition of TPP solution into the chitosan solution under magnetic stirring at 1000 rpm for 30 minutes. Separately, dextrin (0.1 g) was dispersed in 10 mL of deionized water at a concentration of 1.0% (w/v) and gelatinized in a boiling water bath. The solution was then cooled to room temperature. Insulin (500 μg/mL) was added dropwise to the gelatinized dextrin solution, followed by magnetic stirring at 1000 rpm for 1 hour. Further, Pectin (4% w/v) was incorporated into the chitosan-dextrin-insulin mixture using a high-speed homogenizer. The resulting composition was maintained at 4° C. for 48 hours. The final suspension was centrifuged using a cooling centrifuge, and the resulting pellet was lyophilized to obtain the dry powder form of the INS-CsNBs-PD shell.

### Example 3: Morphological Examinations

The surface morphology and particle size of the prepared insulin-loaded chitosan nanobeads with a pectin-dextrin coating (INS-CsNBs-PD shell) were examined using high-resolution transmission electron microscopy (HR-TEM). Imaging was performed using a JEM 1400 instrument (Japan) operated at 300 kV. A drop of the nanoparticle suspension was placed on a film-coated copper grid, stained with a 2% (w/v) aqueous solution of phosphotungstic acid, and allowed to dry to enhance image contrast. The dried sample was then analyzed under HR-TEM.

Further morphological analysis was conducted using a field emission scanning electron microscope (FESEM) equipped with energy-dispersive X-ray microanalysis hardware (Philips-XL30, Netherlands). Samples were prepared by dispersing the nanoparticles in deionized water and diluting the suspension to a 1:5 (v/v) ratio at room temperature.

### Example 4: Determination of Entrapment Efficiency (EE %) and Drug Loading (DL %)

Entrapment efficiency and drug loading of insulin onto the CsNBs-PD shell nanoparticles were determined by centrifuging the nanoparticle suspensions at 16,000 rpm for 30 minutes at 4° C. to separate the supernatant containing unencapsulated insulin. The concentration of free insulin in both centrifuged and non-centrifuged samples was quantified using high-performance liquid chromatography (HPLC). All measurements were conducted in triplicate (n=3). Entrapment efficiency (EE %) and drug loading (DL %) were calculated using the following equations:

$$EE \% = ( \frac{\begin{matrix}Amount of insulin originally taken - \\ Amount of insulin in supernatant\end{matrix}}{Amount of insulin originally taken} \times 100 )$$

$$DL \% = ( \frac{Amount of insulin originally taken}{Amount of CsNBs - PD shell loaded insulin} \times 100 )$$

### Example 5: In Vivo Experimental Design

Male Wistar albino rats (n=36), with an average body weight ranging from 100 grams (g) to 140 g, were obtained from VACSERA, Cairo, Egypt. Animals were housed in a controlled environment within clean, aerated cages, with unrestricted access to food and water. Environmental conditions were maintained at a temperature of 30±4° C., relative humidity of 45±5%, and a 12-hour light/dark cycle. Prior to the commencement of the experiment, all animals underwent a one-week acclimatization period. During this period, animals were provided with water and a standardized basal diet composed of 50% to 52% carbohydrates, 17% protein, 5.0% fat, and other nutrients. All experimental procedures were conducted in accordance with the ethical guidelines established by the institutional animal care and use committee (IACUC) at Beni-Suef University. Ethical approval for the present disclosure was granted under IACUC approval number: BSU-FS-021-128.

### Example 6: Induction of Type 1 Diabetes Mellitus (DM)

Type 1 diabetes mellitus was induced in overnight-fasted male Wistar albino rats by intraperitoneal injection of a single dose of streptozotocin (STZ) at 55 mg/kg body weight. STZ was freshly dissolved in 0.1 M citrate buffer solution, adjusted to pH 4.5 [S. Padugupati, S. Ramamoorthy, K. Thangavelu, D. Sarma, D. Jamadar, Effective dose of streptozotocin to induce diabetes mellitus and variation of biophysical and biochemical parameters in albino wistar rats, J. Clin. Diagn. Res. 15 (10) (2021) BF01-BF05, incorporated herein by reference in its entirety.]. In order to prevent hypoglycaemic shock, 10% glucose solution was provided in the drinking water four hours post-injection.

Diabetes mellitus was confirmed four days after induction by measuring fasting blood glucose levels. Blood glucose levels were assessed using the CERA-CHECK™ 1070 glucometer (Korea) under fasting and postprandial conditions. Rats exhibiting fasting blood glucose levels exceeding 180 mg/dL were classified as diabetic. Body weight and blood glucose levels were monitored weekly for a duration of one month.

### Example 7: Animal Grouping

FIG. 1B illustrates a schematic representation of an animal model and experimental design. Male Wistar albino rats were randomly assigned to six groups, each including six animals. The first group served as the normal control and received only sodium citrate buffer (0.1 M, pH 4.5). The second group functioned as the diabetic control and was administered a single intraperitoneal injection of STZ at a dose of 55 mg/kg. The third group consisted of diabetic rats treated orally with insulin-loaded chitosan nanobeads coated with a pectin-dextrin shell (INS-CsNBs-PD shell), suspended in 0.025 M hydrochloric acid, at a dose of 50 IU/kg, calculated based on the encapsulation efficiency of insulin [C. Damg\acute{ }e, P. Maincent, N. Ubrich, Oral delivery of insulin associated to polymeric nanoparticles in diabetic rats, J. Contr. Release 117 (2) (2007) 163-170, incorporated herein by reference in its entirety.]. The fourth group included diabetic rats treated orally with free insulin at a dose of 10 IU/kg to evaluate the efficacy of the oral administration route. The fifth group served as a blank control and received unloaded CsNBs-PD shell nanoparticles in 0.025 M hydrochloric acid at a dose equivalent to 50 IU/kg. The sixth group comprised diabetic rats treated with subcutaneous insulin injections at a dose of 10 IU/kg [Y. Wang, H. Sun, J. Zhang, Z. Xia, W. Chen, Streptozotocin-induced diabetic cardiomyopathy in rats: ameliorative effect of PIPERINE via Bcl2, Bax/Bcl2, and caspase-3 pathways, Biosci. Biotechnol. Biochem. 84 (12) (2020) 2533-2544, incorporated herein by reference in its entirety.]. All treatments were initiated one week following STZ administration and were continued daily for a duration of four weeks.

### Example 8: Blood Sampling and ELISA Assay

Throughout the experimental period, and on the day preceding sacrifice, blood samples were collected weekly from the lateral tail veins of the rats to measure fasting and postprandial blood glucose levels. Blood glucose levels were determined using the CERA-CHECK™ 1070 glucometer, which has a maximum measurable range of 600 mg/dL. At the conclusion of the experiment, all animals were sacrificed, and blood was immediately collected from the inferior vena cava into vacuum blood collection tubes. The collected blood was centrifuged at 3000 rpm for 10 minutes to separate the serum. Serum samples (n=6) were stored at −20° C. until further analysis.

Serum insulin and C-peptide concentrations were quantified using ultra-sensitive rat insulin and C-peptide ELISA kits (Crystal Chem, Inc., USA), following protocols established by the manufacturer. Levels of interleukin-1ß (IL-1B) and interleukin-6 (IL-6) were measured using enzyme-linked immunosorbent assay kits obtained from Cloud-Clone Corp, USA, in accordance with the provided instructions. Total protein content was determined using Kruger's method with a protein estimation kit supplied by Genei, Bangalore [N. J. Kruger, The Bradford method for protein quantitation, Methods Mol. Biol. 32 (1994) 9-15, incorporated herein by reference in its entirety.].

### Example 9: Preparation of Tissue Homogenate and Oxidative Stress Determination

Pancreatic tissues were excised from all experimental groups, rinsed in liquid nitrogen, and subsequently thawed at −70° C. Tissue samples were dissected and weighed, then homogenized using a mechanical homogenizer to achieve a 10% (w/v) concentration in ice-cold phosphate-buffered saline (PBS, pH 7.4) [A. Ahangarpour, A. A. Oroojan, L. Khorsandi, M. Kouchak, M. Badavi, Solid lipid nanoparticles of myricitrin have antioxidant and antidiabetic effects on streptozotocin-nicotinamide-induced diabetic model and myotube cell of male mouse, Oxid. Med. Cell. Longev. 2018 (2018) 7496936, incorporated herein by reference in its entirety.]. The resulting homogenates were centrifuged at 4000 rpm for 10 minutes, and the supernatants were collected and stored at −20° C. for subsequent biochemical analysis.

Oxidative stress markers were evaluated in the supernatants from each group (n=6). Malondialdehyde (MDA) levels were measured as an indicator of lipid peroxidation [M. C. Sabu, R. Kuttan, Antidiabetic activity of Aegle marmelos and its relationship with its antioxidant properties, Indian J. Physiol. Pharmacol. 48 (1) (2004) 81-88, incorporated herein by reference in its entirety.]. Antioxidant enzyme activities, including catalase (CAT), superoxide dismutase (SOD), glutathione S-transferase (GST), glutathione peroxidase (GPx), and reduced glutathione (GSH), were also quantified [O. O. Erejuwa, S. A. Sulaiman, M. S. Wahab, S. K. Salam, M. S. Salleh, S. Gurtu, Antioxidant protective effect of glibenclamide and metformin in combination with honey in pancreas of streptozotocin-induced diabetic rats, Int. J. Mol. Sci. 11 (5) (2010) 2056-2066, incorporated herein by reference in its entirety].

### Example 10: Histological Examination

Following the fourth week of treatment, all rats were sacrificed by decapitation under light anaesthesia. Pancreatic tissues were immediately dissected and sectioned. Tissue samples were fixed in 10% neutral buffered formalin for 24 hours. Dehydration was performed by passing the fixed tissues through a graded series of alcohol concentrations. Tissues were then cleared by replacing alcohol with xylene and subsequently embedded in paraffin wax at 60° C. Paraffin-embedded pancreatic blocks were sectioned into slices approximately 4 μm thick using a microtome. The resulting tissue sections (n=6) were deparaffinized and stained with hematoxylin and eosin (H&E) for histological evaluation [J. D. Bancroft, M. Gamble, in: Theory and Practice of Histological Techniques 5th, Edinburgh. Churchill Livingstone Pub, 2002, pp. 172-175, 593-175, Am J Cancer Prev. 3 (6) (2015) 122-175, incorporated herein by reference in its entirety.].

### Example 11: Immunohistochemistry and Morphometric Analysis

Insulin granules in pancreatic islets were detected using immunohistochemical staining techniques [S. C. Campbell, W. M. Macfarlane, Detection of insulin production by immunohistochemistry, Methods Mol. Med. 83 (2003) 47-49, incorporated herein by reference in its entirety.]. Paraffin-embedded pancreatic tissue sections, 4 μm in thickness, were rehydrated and mounted on positively charged slides. The sections were washed with 0.1 M phosphate-buffered saline (PBS) and subjected to the immunohistochemical procedure using the Avidin-Biotin detection system (Ventana, Tucson, AZ, USA), in accordance with the manufacturer's instructions (n=6).

In order to eliminate non-specific binding caused by endogenous peroxidase activity, sections were incubated in 3% hydrogen peroxide for 10 minutes, followed by antigen retrieval through boiling in citrate buffer (pH 6.0). A protein block was applied to further reduce non-specific interactions. Primary anti-insulin antibodies (ICBTACLS), biotin-conjugated and supplied by eBioscience™, Thermo Scientific, USA, were applied to the tissue sections for specific detection of insulin. The area percentage of insulin immunoexpression was quantified using ImageJ software (Wayne Rasband, NIH, Bethesda, MD, USA) at a magnification of 400 times. Five distinct, non-overlapping immunostained fields were analyzed per animal. Data were expressed as mean±standard error (SE).

### Example 12: Quantitative Real-Time Polymerase Chain Reaction (qRT-PCR)

Gene expression levels of Bcl2-associated X apoptotic activator (Bax), anti-apoptotic mediator Bcl2 (B-cell lymphoma 2), nuclear factor erythroid 2-related factor 2 (Nrf2), and heme oxygenase-1 (HO-1) were evaluated using quantitative real-time polymerase chain reaction (qRT-PCR). Total RNA was extracted from frozen pancreatic tissue samples (−80° C.) (n=6) using a nucleic acid extraction kit (NucleoSpin®, REF. 740901.250) obtained from Macherey-Nagel GmbH & Co. KG, Germany.

The concentration and purity of the extracted RNA were assessed spectrophotometrically at 260 nm by measuring the A260/A280 ratio using a dual-wavelength Beckman spectrophotometer (USA) [See: D. R. I. Abdel-Gawad, W. A. Moselhy, R. R. Ahmed, H. M. Al-Muzafar, K. A. Amin, M. M. Amin, E. S. Nahass, K. A. H. Abdou, Therapeutic effect of mesenchymal stem cells on histopathological, immunohistochemical, and molecular analysis in second-grade burn model, Stem Cell Res. Ther. 12 (1) (2021) 308, incorporated herein by reference in its entirety.]. Complementary DNA (cDNA) was synthesized from the RNA samples using reverse transcriptase. Amplification of cDNA was performed using SYBR™ Green (SensiFAST™) PCR Master Mix (Thermo Scientific, USA) [J. W. Hwang, S.-N. Kim, N. Myung, D. Song, G. Han, G.-U. Bae, M. T. Bedford, Y. K. Kim, PRMT5 promotes DNA repair through methylation of 53BP1 and is regulated by Src-mediated phosphorylation, Commun. Biol. 3 (1) (2020) 428, incorporated herein by reference in its entirety.]. Primer sequences used for amplification are listed in Table 1.

TABLE 1
Primer sequence (forward and reverse) for qRT-PCR.

| Gene | Gene accession number | Primer sequence (5′→3′) | Reference |
|---|---|---|---|
| Bax | U32098.1 | F: CCTGAGCTGACCTTGGAGCA | *1 |
| | | R: GGTGGTTGCCCTTTTCTACT | |
| Bc12 | NM_016993.1 | F: TGATAACCGGGAGATCGTGA | *2 |
| | | R: AAAGCACATCCAATAAAAAGC | |
| Nrf2 | NM_031789.2 | F: TCCCAAACAAGATGCCTTGT | *3 |
| | | R: AGAGGCCACACTGACAGAGA | |
| HO-1 | NM_012580 | F: CACCAGCCACACAGCACTAC | *4 |
| | | R: CACCCACCCCTCAAAAGACA | |
| GAPDH | NM_017008.4 | F: GGATACTGAGAGCAAGAGAGA | *5 |
| | | R: TTATGGGGTCTGGGATGGAA | |

Table 1 includes the following references, all incorporated herein by references in their entireties:
*1 L.S. Binmahfouz, B.G. Eid, A.M. Bagher, R.A. Shaik, N.S. Binmahfouz, A.B. Abdel-Naim, Piceatannol SNEDDS attenuates estradiol-induced endometrial hyperplasia in rats by modulation of NF-κB and Nrf2/HO-1 axes, Nutrients 14 (9) (2022) 1891.
*2 H. Almukadi, B.G. Eid, R.A. Shaik, A.B. Abdel-Naim, A. Esmat, Auraptene nanoparticles ameliorate testosterone-induced benign prostatic hyperplasia in rats: emphasis on antioxidant, anti-inflammatory, proapoptotic and PPARs activation effects, Biomed. Pharmacother. 143 (2021) 112199.
*3 A.R. Ayob, A.H. Al-Najjar, A.S. Awad, Amelioration of bile duct ligation induced liver injury by lactoferrin: role of Nrf2/HO-1 pathway, AIJPMS 2 (2021) 84-90.
*4 A. Bagalagel, R. Diri, A. Noor, D. Almasri, H.T. Bakhsh, H.I. Kutbi, M.M.H. Al-Gayyar, Curative effects of fucoidan on acetic acid induced ulcerative colitis in rats via modulating aryl hydrocarbon receptor and phosphodiesterase-4, BMC Complement Med Ther 22 (1) (2022) 1-12
*5 C.A. Kelm-Nelson, S.A. Stevenson, M.R. Ciucci, Data in support of qPCR primer design and verification in a Pink1 -/- rat model of Parkinson disease, Data Brief 8 (2016) 360-363.

Real-time PCR was conducted using the StepOne Real-Time PCR System (Applied Biosystems, Foster City, USA). Gene expression data were analyzed using the (2\hat{ }−\Delta\Delta Ct}) method. Expression levels of each target gene were normalized to the reference gene glyceraldehyde-3-phosphate dehydrogenase (GAPDH) and expressed as a percentage relative to the control group.

### Example 13: Western Blot (WB) Assay

Total protein was extracted from homogenized pancreatic tissue samples (n=6) using the ReadyPrep™ protein extraction kit (total protein) from Bio-Rad Inc. (Catalog #163-2086), following the protocols established by the manufacturer. Protein concentrations were quantified using the Bradford Protein Assay Kit (SK3041) supplied by Bio Basic Inc., Markham, Ontario, Canada.

A 20 μg aliquot of protein from each sample was separated by sodium dodecyl sulfate polyacrylamide gel electrophoresis (SDS-PAGE) using the TGX Stain-Free™ Fast Cast™ acrylamide kit (Bio-Rad Laboratories Inc., Cat #161-0181). Proteins were transferred onto polyvinylidene difluoride (PVDF) membranes and blocked in tris-buffered saline with Tween 20 (TBST) containing 3% bovine serum albumin (BSA) to prevent non-specific antibody binding [B. F. Brian, C. R. Guerrero, T. S. Freedman, Immunopharmacology and quantitative analysis of tyrosine kinase signalling, Curr. Protoc. Im. 130 (1) (2020) e104, incorporated herein by reference in its entirety.].

Detection of target proteins was performed in triplicate by incubation with primary antibodies, followed by incubation with appropriate secondary antibodies. The primary antibodies used were NF-κB p65 (F-6): sc-8008 and anti-SIRT1 (B-7): sc-74465. Protein expression levels were normalized to B-actin, which served as the internal loading control. The anti-β-actin antibody [(C-2): sc-8432] was obtained from Santa Cruz Biotechnology, Inc., CA, USA [T. Mahmood, P. C. Yang, Western blot: technique, theory, and trouble shooting, N. Am. J. Med. Sci. 4 (9) (2012) 429-434, incorporated herein by reference in its entirety.]. Immunoreactive bands were visualized using an electrochemiluminescence (ECL) detection system (Pierce, Rockford, IL, USA). The chemiluminescent substrate used was Clarity™ western ECL substrate (Bio-Rad, Cat #170-5060).

### Example 14: Statistical Studies

All quantitative data were expressed as mean±standard error (SE). Statistical analysis was performed using IBM SPSS statistics version 17 (IBM Corp., NY, USA). One-way analysis of variance (ANOVA) was employed to assess differences among groups, followed by a post hoc test for multiple comparisons. A p-value less than or equal to 0.05 (P<0.05) was considered indicative of statistical significance. In addition to statistical summaries, individual data points were visually represented as scattered dots on the corresponding graphs to illustrate data distribution within each group.

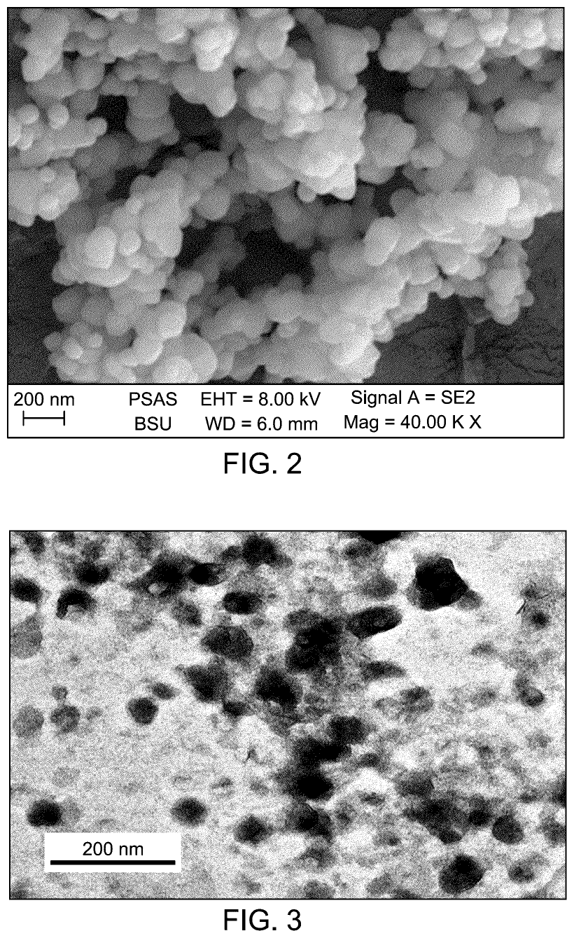

FIG. 2 illustrates high-resolution transmission electron micrographs (HRTEM) of insulin-loaded chitosan nanobeads coated with a pectin-dextrin shell (INS-CsNBs-PD nanoparticles). The HRTEM images reveal that the nanoparticles are approximately spherical in shape, with an average particle diameter of 92 nm.

FIG. 3 illustrates field emission scanning electron microscopy (FESEM) images of the same nanoparticles. The FESEM analysis confirms the spherical morphology and shows that the particles possess smooth surfaces and exhibit relatively uniform size and shape. The mean particle diameter observed in the FESEM images is approximately 100 nm. No significant difference in particle size was observed between the HRTEM and FESEM analyses.

Based on Equation 1 and equation 2, the encapsulation efficiency of insulin-loaded chitosan nanobeads coated with a pectin-dextrin shell (INS-CsNBs-PD shell nanoparticles) was determined to be 69.3±2.75% (n=3), while the drug loading was calculated as 26.28=0.56% (n=3). The aforementioned values confirm the successful incorporation of insulin into the nanoparticle matrix and demonstrate the composition's capacity for efficient drug loading.

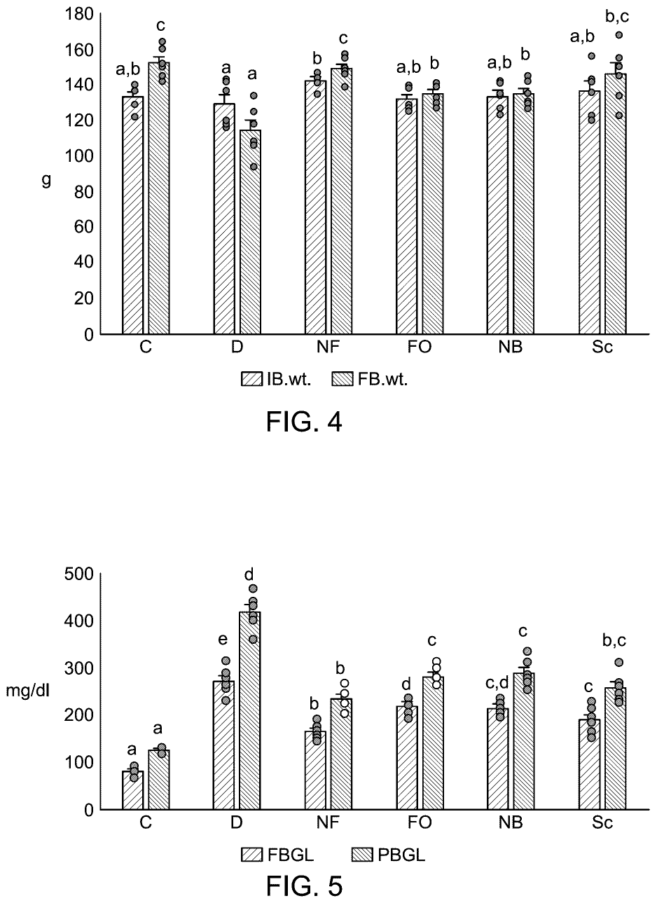

FIG. 4 illustrates initial and final body weights of rats across the various treatment groups. At the beginning of the examinations described in the present disclosure, no significant differences in body weight were observed among the groups. However, by the end of the four-week treatment period, the untreated diabetic group exhibited a significant reduction in body weight compared to the normal control group (P<0.05). Treatment with either orally administered insulin-loaded chitosan nanobeads coated with a pectin-dextrin shell (INS-CsNBs-PD shell) or subcutaneously injected insulin significantly improved body weight in diabetic rats (P<0.05). No statistically significant difference was observed between the effects of the oral and subcutaneous insulin treatments. The use of insulin-loaded chitosan nanoparticles with a protective coating contributed to the restoration of body weight in rats with STZ-induced diabetes.

FIG. 5 presents the fasting and postprandial blood glucose levels (FBS and PBS) across the experimental groups. Four weeks following STZ administration, the untreated diabetic group exhibited significantly elevated FBS and PBS levels compared to the healthy control group (P<0.05). Oral administration of the INS-CsNBs-PD shell composition resulted in a significant reduction in both FBS and PBS levels, comparable to the effects observed with subcutaneous insulin injections (P<0.05). Moreover, the insulin-loaded CsNBs-PD shell demonstrated a greater potential to reduce blood glucose levels than either free oral insulin or subcutaneous insulin. The observed hypoglycaemic effect was attributed to the synergistic action of chitosan and pectin in enhancing insulin stability and absorption.

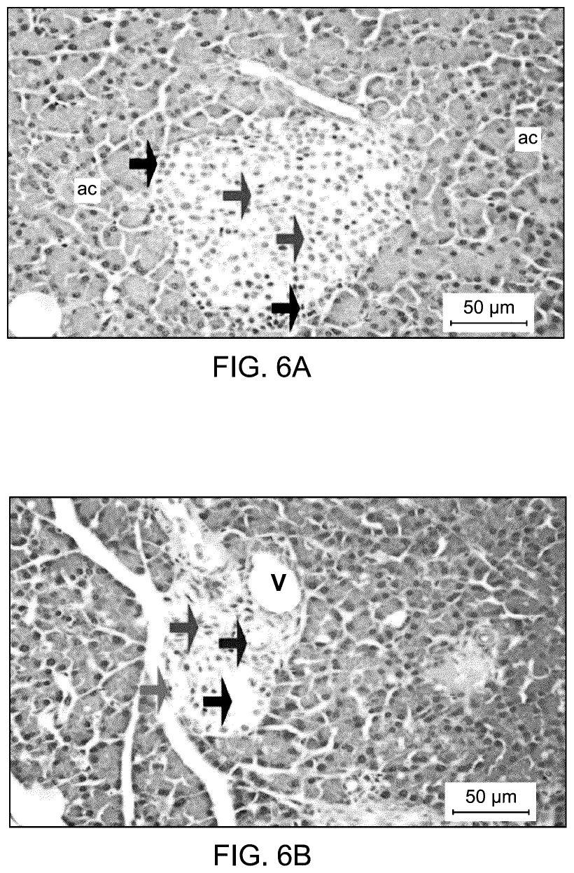

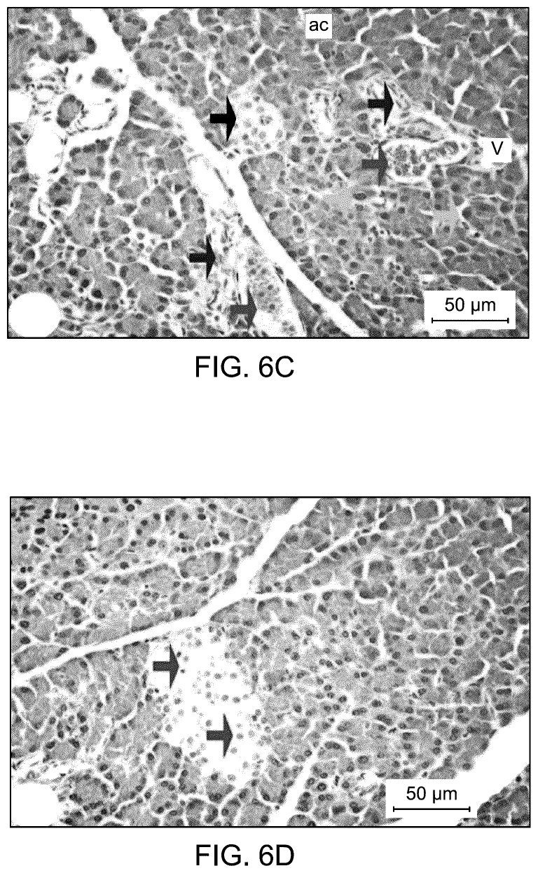

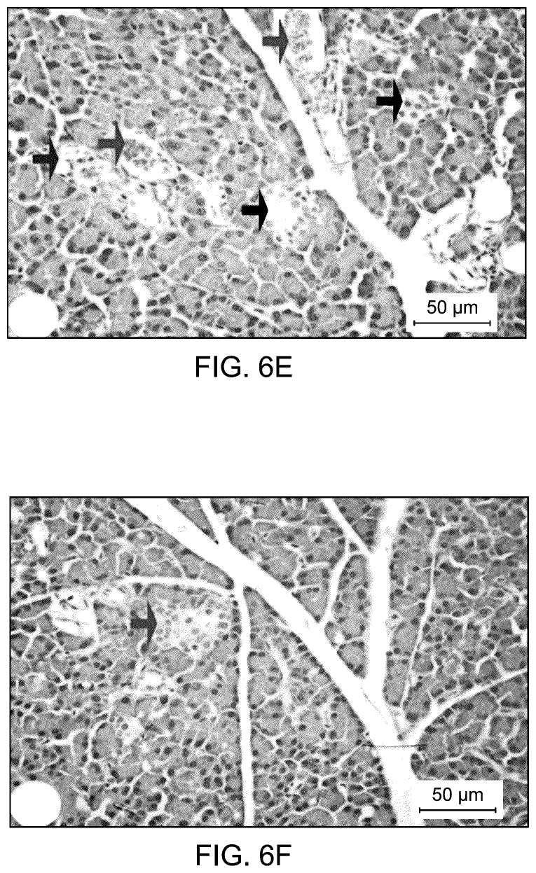

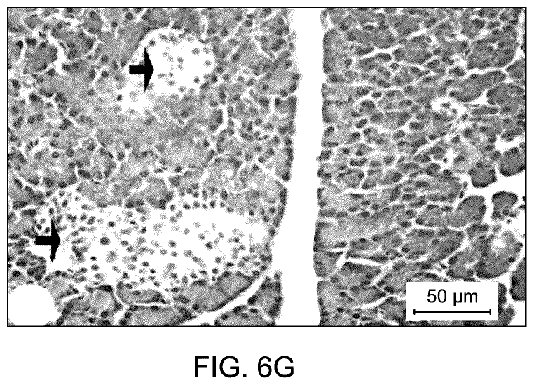

FIGS. 6A-6G illustrate the histological architecture of pancreatic tissues across the experimental groups. In the normal control group, pancreatic sections exhibited well-preserved cytoplasm and nuclei, with pancreatic acini appearing oval or rounded in shape. The acini were composed of pyramidal cells arranged around a small acinar lumen and contained zymogen granules in their cytoplasm. Connective tissue septa between the acini were minimal. The islets of Langerhans appeared as pale, non-encapsulated, round or oval clusters composed of irregular, branching, and anastomosing cords of cells separated by blood capillaries, as shown in FIG. 6A. In contrast, pancreatic tissues from the STZ-induced diabetic group showed significant pathological alterations. The islets of Langerhans were reduced in size and number, with some sections exhibiting complete degeneration. Central regions of the islets displayed marked cellular degeneration, including cytoplasmic vacuolation, necrosis, and pyknotic nuclei, as shown in FIG. 6B. Additional sections revealed shrunken islet cells with acidophilic, vacuolated cytoplasm and congested blood vessels, as shown in FIG. 6C. Further, oral administration of the INS-CsNBs-PD shell composition resulted in substantial histological improvement. Numerous islets of varying sizes were observed, with evidence of reduced degenerative changes and limited β-cell regeneration, as shown in FIG. 6D. In contrast, diabetic rats treated with free oral insulin exhibited decreased islet volume and congested blood capillaries, with minimal histological recovery and limited improvement in oxidative stress markers, as shown in FIG. 6E. Subcutaneous insulin treatment led to notable restoration of islet architecture, with increased islet size and only a few hyperchromatic nuclei remaining, as shown in FIG. 6G.

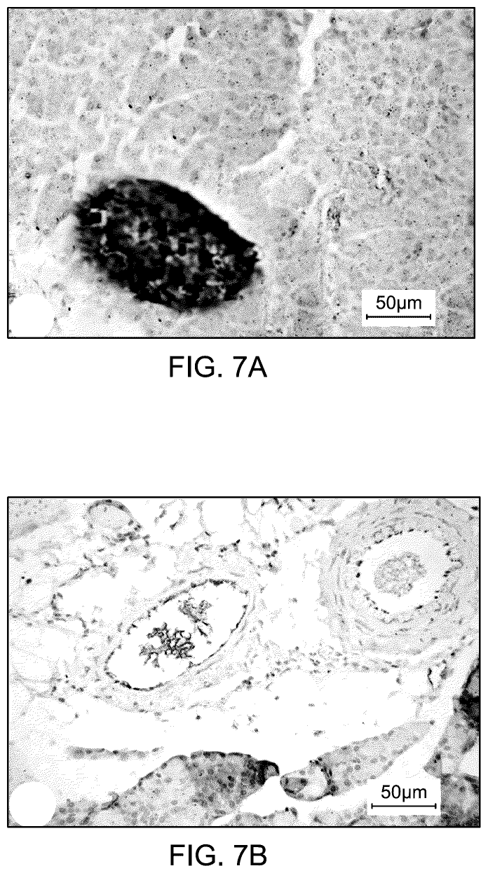

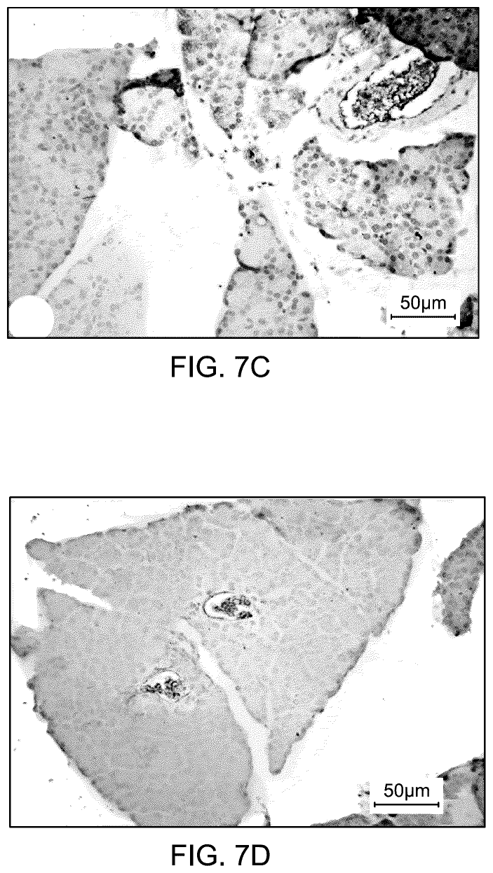

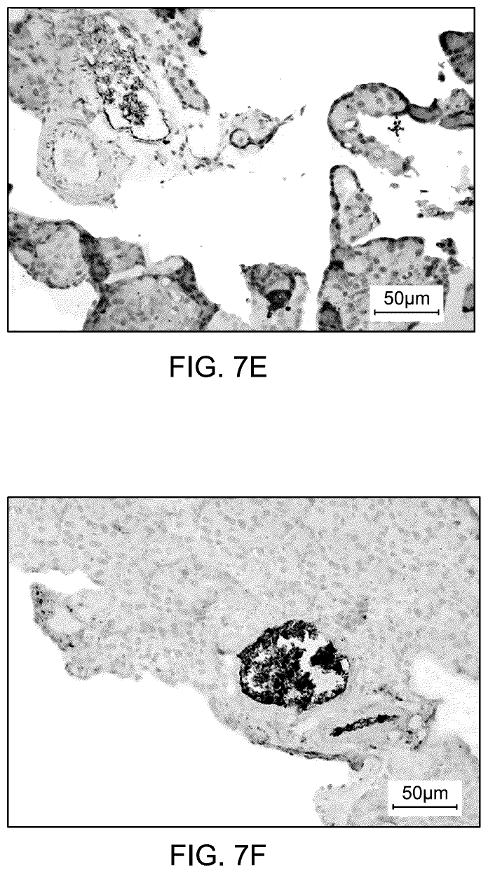

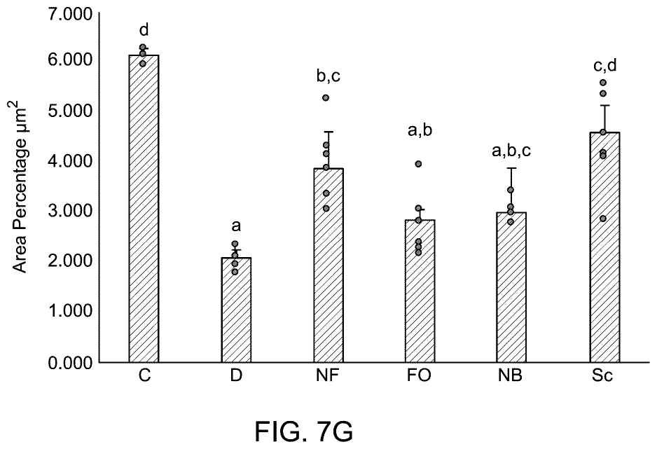

The core region of the pancreatic islets was predominantly occupied by β-cells, or insulin-secreting cells, which constituted the majority of the islet cell population. These β-cells were identified by the presence of dark brown cytoplasmic granules, indicative of normal positive insulin expression, as β-cells treated with anti-insulin antibodies exhibited a strong positive immunoreactive response, as shown in FIG. 7A. In the diabetic group, the immunological response to anti-insulin antibodies was markedly diminished due to a reduction in insulin-positive β-cells and disruption in their distribution, as shown in FIG. 7B. Following oral administration of the INS-CsNBs-PD shell composition, insulin expression remained minimal, corresponding to a limited number of β-cells, as shown in FIG. 7C. Furthermore, pancreatic sections from rats treated with free oral insulin displayed small islets, a substantial loss of β-cells, and an immunohistochemical insulin expression pattern similar to that observed in the untreated diabetic group, as shown in FIG. 7D. Administration of unloaded CsNBs-PD shell nanoparticles also resulted in a significant reduction in insulin expression, attributed to the presence of only a few normal β-cells, as shown in FIG. 7E. In contrast, pancreatic sections from diabetic rats treated with subcutaneous insulin injections exhibited a marked increase in insulin-positive β-cells, with a distribution and density comparable to that of the normal control group, as shown in FIG. 7F.

In diabetic rats, both the free oral insulin-treated group (FO) and the group treated with 50 IU/kg of the CsNBs-PD shell nano-formula exhibited significantly reduced percentage areas of reactive islet β-cells compared to the control group (C). Compared to the untreated diabetic group (D), the subcutaneous insulin group (Sc) (10 IU/kg) and the INS-CsNBs-PD shell oral formula group (NF) (50 IU/kg) demonstrated a significant increase in morphometric measurements, as shown in FIG. 7G.

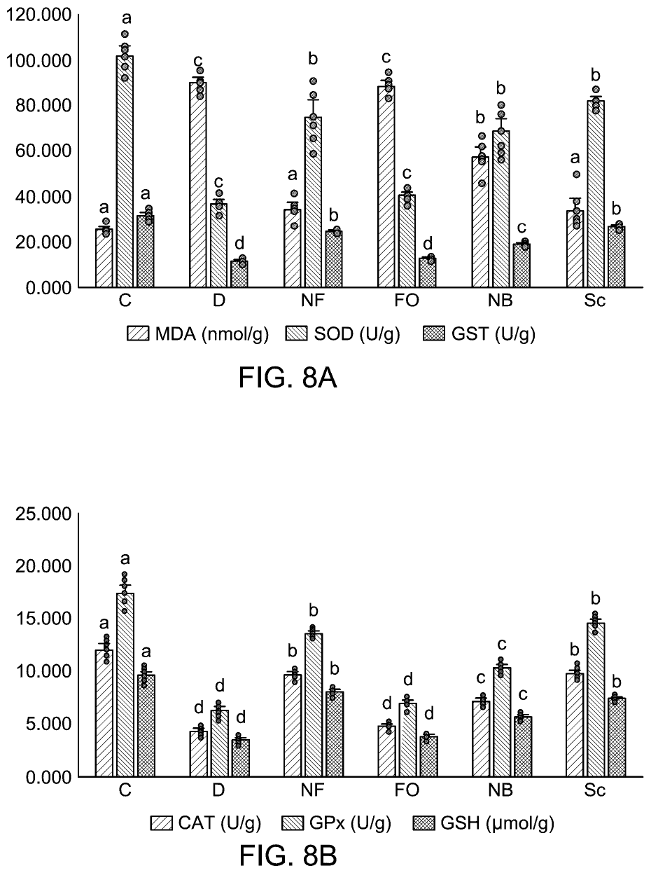

FIGS. 8A-8B illustrate the outcomes of oxidative stress biomarkers. Diabetes significantly reduced the levels of SOD, CAT, GSH, GST, and GPx (P<0.05). Malondialdehyde (MDA) levels were significantly elevated in STZ-injected animals compared to normal control rats. Treatment with orally coated forms of INS-loaded CsNBs-PD and non-coated subcutaneous insulin showed no discernible differences between them. Both treatments significantly reduced oxidative stress by elevating SOD, CAT, GSH, GST, and GPx levels and lowering MDA levels (P<0.05). The group treated with the oral CsNBs-PD shell nano-formula exhibited a significant reduction in MDA and an increase in antioxidant levels, attributed to the presence of chitosan and pectin.

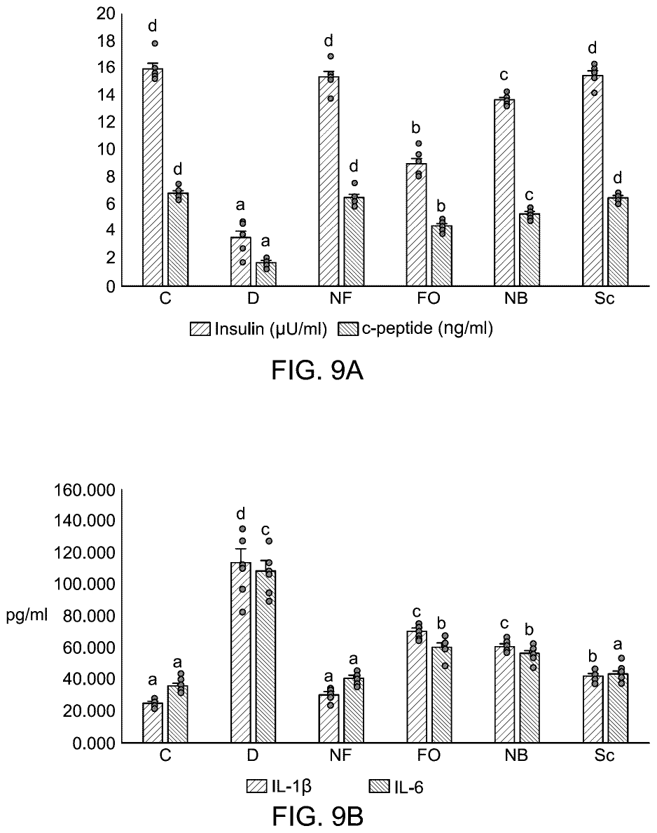

FIG. 9A illustrates the serum insulin and C-peptide levels in normal control and STZ-induced diabetic rats. Compared to normal rats, STZ-induced rats exhibited a significant reduction (P<0.05) in serum insulin and C-peptide levels. In diabetic rats, serum levels of insulin and C-peptide were significantly elevated (P<0.05) following oral administration of the INS-CsNBs-PD shell nano-formula, with effects comparable to subcutaneous insulin. Subsequent increases in serum insulin and C-peptide levels were also observed in the diabetic rat groups treated with the oral CsNBs-PD shell nano-formula (NB) and free oral insulin (FO) (P<0.05). The oxidative stress-induced inflammatory response in STZ-induced diabetic rats resulted in significantly increased levels of IL-1ß and IL-6, as determined by ELISA analysis of serum pro-inflammatory cytokines (P<0.05). Oral treatment with the INS-CsNBs-PD shell nano-formula restored IL-1B and IL-6 levels to near-normal values compared to the normal control group. Following subcutaneous insulin injection and oral administration of CsNBs-PD nanoparticles, IL-1ß and IL-6 levels were significantly reduced compared to the STZ-diabetic group (P<0.05), as shown in FIG. 9B.

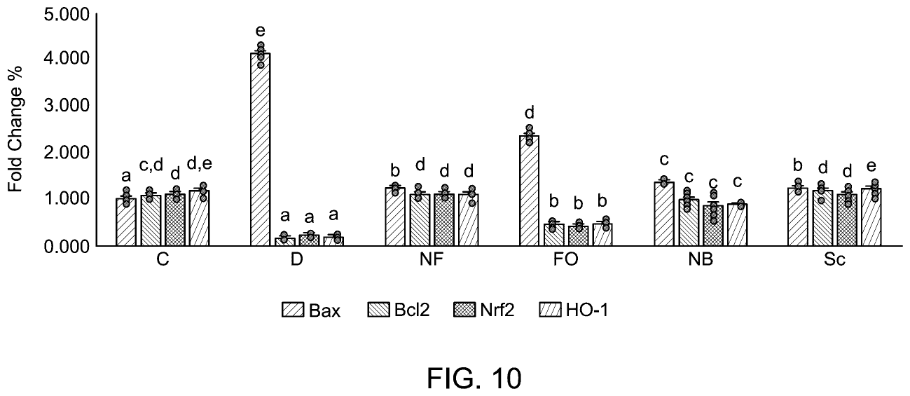

Statistical analysis revealed that gene expression of Bax was significantly increased in diabetic rats, while the expression of Bcl-2, Nrf2, and HO-1 was significantly decreased (P<0.05). Compared to the control group, both the diabetic group and the diabetic rats administered free oral insulin exhibited significantly higher Bax mRNA expression. However, Bax expression was significantly lower in rats treated with subcutaneous insulin and in those receiving oral nano-compositions of INS-CsNBs-PD shell and CsNBs-PD shell nanoparticles. In comparison to the diabetic group, rats treated with the oral nano-formula of INS-CsNBs-PD shell NPs, CsNBs-PD shell NPs, and subcutaneous insulin injections showed significant improvements in the gene expression of Bcl-2, Nrf2, and HO-1 (P<0.05), as shown in FIG. 10.

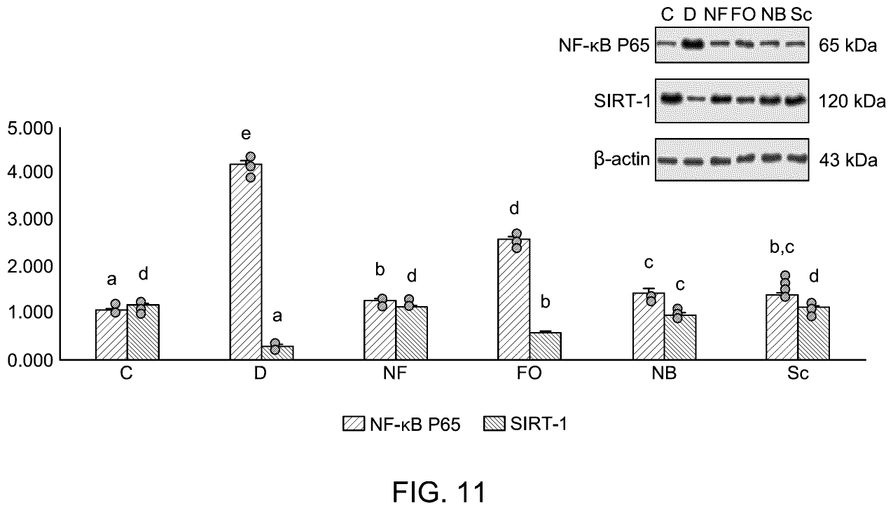

In order to further examine the regulatory roles of NF-κB p65 and SIRT-1, the effects of the INS-CsNBs-PD shell nano-formula on the expression of these proteins were evaluated in STZ-induced diabetic rats. The results demonstrated that oral administration of the INS-CsNBs-PD shell nano-formula, as well as subcutaneous insulin treatment, significantly increased the expression of SIRT-1 protein and decreased the expression of NF-κB p65 protein compared to the diabetic group (P<0.05). Additionally, oral treatment with unloaded CsNBs-PD shell nanoparticles markedly enhanced SIRT-1 expression and reduced NF-κB p65 expression relative to both the diabetic and control groups, as shown in FIG. 11.

To conclude, the present provides an oral insulin delivery composition including insulin-loaded chitosan nanobeads encapsulated within a pectin-dextrin shell (INS-CsNBs-PD shell), formulated for administration in subjects exhibiting streptozotocin (STZ)-induced diabetic conditions. The disclosed composition has been examined for its ability to promote sustained insulin release, modulate blood glucose levels, and exert antioxidative and anti-apoptotic effects in pancreatic tissue. The composition was observed to influence pancreatic histology by preserving β-cell morphology and functionality.

Oral administration of the disclosed composition resulted in significant modulation of biochemical and histological markers, including decreased blood glucose levels, enhanced pancreatic B-cell architecture, and increased expression of insulin-immunopositive β-cells. The composition elevated the activity of antioxidant enzymes including superoxide dismutase (SOD), catalase (CAT), glutathione (GSH), glutathione-S-transferase (GST), and glutathione peroxidase (GPx), while concurrently reducing malondialdehyde (MDA) levels. These effects are indicative of a reduction in oxidative stress and lipid peroxidation.

Gene and protein expression analysis demonstrated increased Bcl-2 mRNA levels, reduced Bax transcription, and elevated expression of SIRT1 protein in pancreatic tissues following administration of the disclosed composition. The composition further upregulated nuclear factor erythroid 2-related factor 2 (Nrf2) and heme oxygenase-1 (HO-1), consistent with enhanced antioxidative gene expression. Immunohistochemical examination revealed an increase in β-cell insulin reactivity and preservation of islet histoarchitecture. Additionally, a significant decrease in proinflammatory cytokines including interleukin-1ß (IL-1ß) and interleukin-6 (IL-6) was observed, indicating attenuation of inflammatory signaling pathways within pancreatic tissues.

The disclosed composition demonstrated the ability to modulate apoptotic signalling pathways through the suppression of NF-κB P65 activity via the upregulation of SIRT1, and the stabilization of mitochondrial apoptotic regulators, thereby mitigating β-cell apoptosis. Enzymatic analyses further confirmed the restoration of redox balance in pancreatic tissues through increased enzymatic antioxidant activity and reduction in lipid peroxidation markers.

Collectively, the present disclosure establishes that the INS-CsNBs-PD shell composition confers therapeutic effects in STZ-induced diabetic conditions by providing glycaemic regulation, preserving pancreatic β-cell architecture, enhancing antioxidant enzyme profiles, modulating pro-apoptotic and anti-apoptotic gene expression, and suppressing inflammatory and oxidative stress pathways at the molecular and tissue levels.

Numerous modifications and variations of the present disclosure are possible in light of the above teachings. It is therefore to be understood that within the scope of the appended claims, the invention may be practiced otherwise than as specifically described herein.

1. A pharmaceutical composition, comprising
therapeutic nanoparticles comprising
a core including insulin,
a shell comprising chitosan, the shell disposed on the core, and
a coating comprising pectin and dextrin disposed on the shell; and
a pharmaceutically acceptable carrier and/or excipient.

2. The pharmaceutical composition of claim 1, wherein the nanoparticles have a mean particle size of 25 to 250 nm.

3. The pharmaceutical composition of claim 1, wherein the core further includes dextrin.

4. The pharmaceutical composition of claim 1, wherein the dextrin is dextrin derived from corn having a reducing sugar content of less than 5 wt. % based on a total weight of dextrin.

5. The pharmaceutical composition of claim 1, wherein the pectin is pectin derived from citrus peel having a galacturonic acid of at least 74.0 wt. % based on a total weight of pectin.

6. The pharmaceutical composition of claim 1, wherein the chitosan has a mean molecular weight of 190,000 to 300,000 Da.

7. The pharmaceutical composition of claim 1, wherein the pharmaceutically acceptable carrier and/or excipient is at least one selected from the group consisting of a buffer, an inorganic salt, a synthetic fatty acid, a vegetable oil, a fatty ester, a surfactant, and a polymer.

8. A method of forming the pharmaceutical composition, the method comprising:
adding a sodium tri-polyphosphate solution to a chitosan solution comprising the chitosan and acetic acid while stirring to form a shell solution;
mixing a dextrin solution with an insulin solution to form a core solution;
forming a pectin solution;
mixing the shell solution, the core solution, and the pectin solution using a homogenizer to form a precursor mixture;
aging the precursor mixture at 4° C. for 24 to 72 hours to form the nanoparticles; and
mixing the nanoparticles with the pharmaceutically acceptable carrier and/or excipient.

9. The method of claim 8, wherein the dextrin solution is formed by heating a mixture of dextrin and water to 100° C. and cooling to room temperature.

10. The method of claim 8, wherein the chitosan solution has a chitosan concentration of 0.1 to 1% w/v and a pH of 3.5 to 6.0.

11. The method of claim 8, wherein the dextrin solution has a dextrin concentration of 0.25 to 2.5% w/v.

12. The method of claim 8, wherein the insulin solution has an insulin concentration of 100 to 1000 μg/mL.

13. The method of claim 8, wherein the pectin solution has a pectin concentration of 2.5 to 7.5 w/v.

14. The method of claim 8, wherein the method has an insulin encapsulation efficiency of 60 to 80% based on a total amount of insulin present in the core solution.

15. The method of claim 8, wherein the method has an insulin drug loading of 17.5 to 37.5% based on a total amount of insulin present in the core solution.

16. The method of claim 8, wherein the pharmaceutically acceptable carrier and/or excipient is at least one selected from the group consisting of a buffer, an inorganic salt, a synthetic fatty acid, a vegetable oil, a fatty ester, a surfactant, and a polymer.

17. A method of treating diabetes, the method comprising
administering to a subject in need thereof an effective amount of the pharmaceutical composition of claim 1.

18. The method of claim 17, wherein the diabetes is at least one selected from the group consisting of STZ-induced diabetes and Type 1 diabetes.

19. The method of claim 17, wherein the pharmaceutical composition is administered in an amount of 10 to 100 IU of nanoparticles per kg of bodyweight.

20. The method of claim 17, wherein the pharmaceutical composition is administered orally.
